(19) United States
(12) Patent Application Publication
(10) Pub. No.: US 2020/0197554 A1
(43) Pub. Date: Jun. 25, 2020

(54) DISTRIBUTION SYSTEM FOR OZONE

(71) Applicant: Brendon Ray SPENCER, Christchurch (NZ)

(72) Inventor: Brendon Ray SPENCER, Christchurch (NZ)

(21) Appl. No.: 16/226,700

(22) Filed: Dec. 20, 2018

(51) Int. Cl. A61L 2/20; A61L 9/015

(52) U.S. Cl. CPC A61L 2/202; A61L 9/015; A61L 2209/212; A61L 2209/111; A61L 2202/15

(57) ABSTRACT

An ozone distribution system wherein an ozone generator of known type in use supplies ozone at a preselected concentration to an ozone distribution pipe which is arranged to pass through or around the area to be treated with ozone, said ozone distribution pipe being arranged to provide a continuous circuit and to provide a smooth path without sharp bends, said ozone distribution pipe being perforated at intervals along its length, to allow ozone to disperse from the pipe.

### TECHNICAL FIELD

The present invention relates to a system for distributing ozone (tri-oxygen) gas in large quantities and for extended periods, for the purposes discussed below.

### BACKGROUND ART

Ozone is a powerful oxidant, and has a range of uses, predominantly in industrial, agricultural and medical applications in sanitising and deodorising. Ozone also is known to be of use in killing microorganisms in air and water.

Ozone's usefulness has two major limitations:—first, it is an unstable molecule, which readily decomposes into oxygen under normal atmospheric conditions. This means that, unlike most industrial gases, ozone cannot be generated and stored for later use, but must be produced on site for immediate use.

Second, ozone is toxic to human beings and can cause damage to mucus and respiratory tissues in human beings and animals, above concentrations of 0.1 parts per million. It follows that if ozone is used anywhere where human beings may also be present, the levels of ozone must be monitored, to ensure that no one is harmed. However, the rapid decomposition rate of ozone means that it is relatively easy to control its concentration in any given location, because providing the continued generation of ozone from the generating device ceases, the natural decomposition of ozone into harmless oxygen clears any undesirably high concentration relatively rapidly.

Ozone can be generated by a number of commercially available devices, the commonest of which are listed below:

1. Corona discharge equipment is used to generate ozone from ambient air, and can produce ozone in a concentration of 3-6 percent.

2. UV ozone generators also generate ozone from ambient air, and typically produce a concentration of about 0.5 percent.

3. Cold plasma machines use pure oxygen to generate ozone and can produce concentrations of up to 5 percent ozone.

4. Electrolytic ozone generation produces hydrogen, oxygen and ozone from water.

In this specification, ozone generating devices are not described in detail, given that any of a wide range of commercially available ozone generating devices may be used in the present invention.

Ozone distribution systems for distributing ozone around a building such as a cool store, to keep stored fruit or vegetables fresh and/or provide control of bacteria and moulds, are known, but require a high rate of ozone generation because of the rapid decomposition of ozone in the system; this increases the operating expense of the system.

### DISCLOSURE OF INVENTION

An object of the present invention is the provision of an ozone distribution system capable of overcoming at least some of the above described drawbacks.

The present invention provides an ozone distribution system wherein an ozone generator of known type in use supplies ozone at a preselected concentration to an ozone distribution pipe which is arranged to pass through or around the area to be treated with ozone, said ozone distribution pipe being arranged to provide a continuous circuit and to provide a smooth path without sharp bends, said ozone distribution pipe being perforated at intervals along its length, to allow ozone to disperse from the pipe.

As used herein, the term “sharp bend” means a bend through an angle equal to or less than 90 degrees. Preferably, all of the angles between adjacent sections of pipe are greater than 90 degrees, most preferably at least 130 degrees.

### BRIEF DESCRIPTION OF THE DRAWINGS

By way of example only, a preferred embodiment of the present invention is described in detail, with reference to the accompanying drawings in which:—

FIG. 1 is a diagrammatic representation of a typical prior art ozone distribution system;

FIG. 2 is a diagrammatic representation of a first ozone distribution system in accordance with the present invention;

FIG. 3 is a diagrammatic representation of a second ozone distribution system in accordance with the present invention; and

FIG. 4 is a diagram showing a typical control sequence for an ozone distribution system in accordance with the present invention.

### BEST MODE FOR CARRYING OUT THE INVENTION

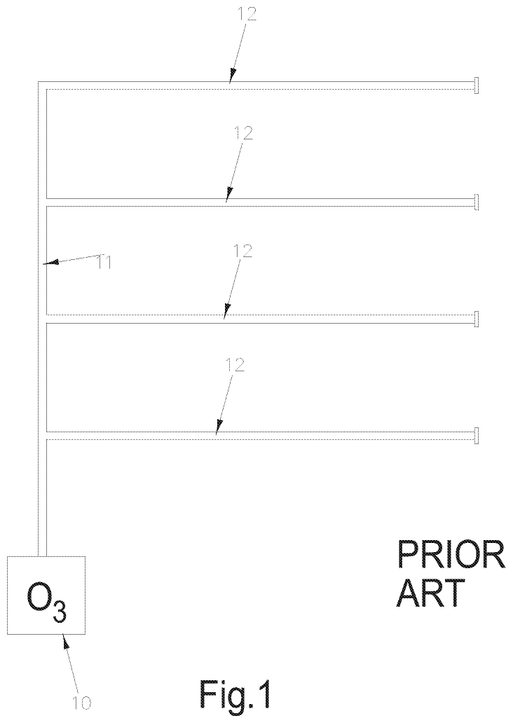

FIG. 1 shows a typical prior art ozone distribution system:—ozone is produced by a known commercially available ozone generator 10 and ozone is supplied to an outflow pipe 11 to each of a plurality of closed ended parallel pipes 12. Each of the pipes 12 is perforated, so that ozone can pass out of each pipe and into the surrounding air. The use of closed pipes for distribution tends to cause a back pressure in the system, which reduces the overall efficiency of the system. Further, the rate of ozone decomposition is high:—typically, in the distribution system shown in FIG. 1, forcing the ozone around a sharp (90°) bend from the pipe 11 into the pipes 12 can reduce the ozone level by 50%. In systems of this type, a generated ozone concentration of 250 ppb (parts per billion) can be reduced to 10 ppb or less by the end of the distribution system.

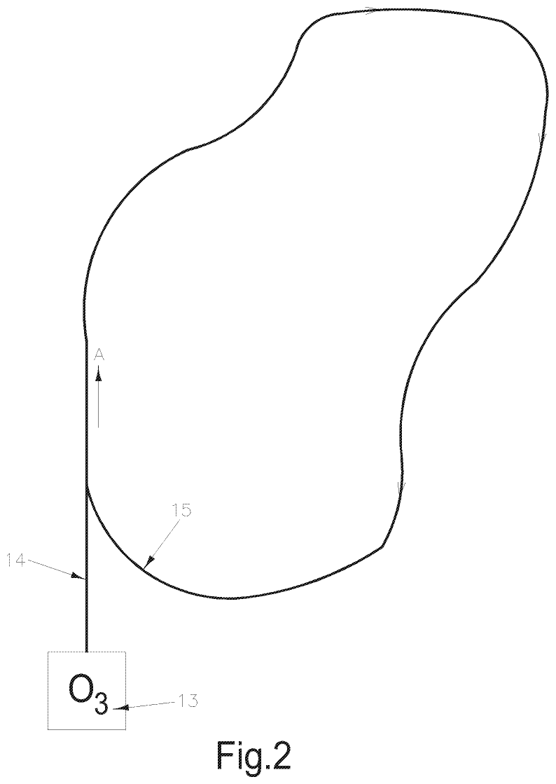

FIG. 2 shows (diagrammatically) an ozone distribution system in accordance with a first embodiment of the invention. In this system, an ozone generator 13 of known type is connected via an outflow pipe 14 to a closed circuit distribution pipe 15, only one loop of which is depicted. The distribution pipe is shaped to as so as to avoid any tight changes of angle or sharp bends, and has no blocked ends:—the ozone produced by the generator 13 travels in the direction of arrow A around a continuous circuit of perforated pipe 15 without sudden changes of direction and passes out of the pipe 15 through a plurality of perforations in the pipe wall. Typically all of the bends are at least 130°. The perforations may be regular or irregular and may be concentrated in selected areas to increase the concentration of ozone in those areas if necessary. The continuous distribution path minimises any back pressure in the system and significantly reduces the rate of ozone decomposition:—it has been found possible to generate and deliver a constant level of 50-80 ppb of ozone in a system of this type, without any significant decomposition losses.

The distribution path provided by pipe 15 is shaped so as to approximately mimic the shape of the area to be treated. It will be appreciated that the shape of the distribution path can be varied to suit any space, provided always that sharp bends are avoided, and a continuous circuit distribution path is maintained.

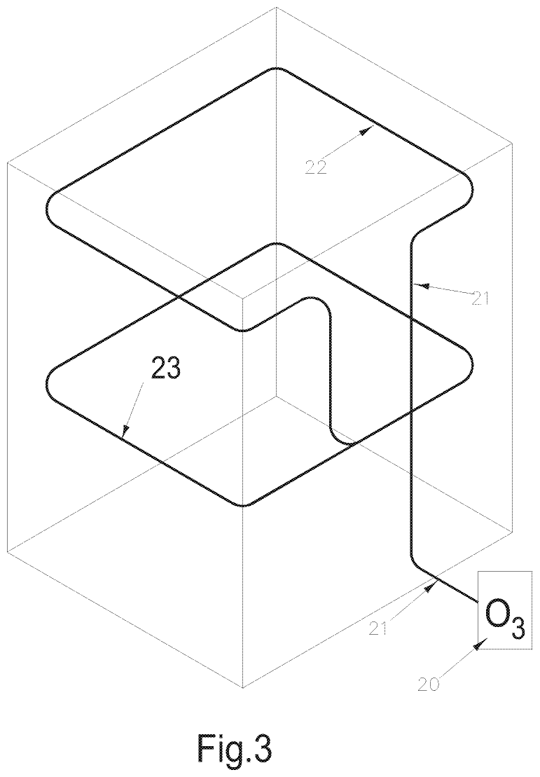

FIG. 3 shows a two-level system, with an ozone generator 20 of known type supplying ozone into an outlet pipe 21 which extends up to the ceiling of the area to be treated and provides a first circuit 22 at the ceiling level, which is connected to a second lower circuit 23 around the walls of the area. As with the FIG. 2 embodiment, the distribution circuits avoid any sharp bends or sudden changes of direction, and provide a continuous circuit distribution. The circuits 22, 23 are made of perforated pipe and, as with the FIG. 2 embodiment, ozone leaves the pipe through the perforations to disperse around the area being treated.

Typically, the outlet and distribution pipes are readily available low-pressure polyvinylchloride pipe of circular cross-section. Typical pipe diameters are in the range 50-100 millimetres. The perforated pipes 15, 22, 23 are perforated with 3 millimetre—5 millimetre holes, which may be single holes or may be on opposite sides of the pipe. The holes may be grouped into selected areas or may be regularly distributed along the length of the pipe e.g. every 500 millimetres.

To provide effective ozone treatment for a shed or similar area approximately 100 by 30 by 10 metres an ozone generator capable of producing about 18-22 cubic millimetres of ozone per hour, distributed through 300 metres of perforated pipe, has been found satisfactory.

The objective of the design of the distribution path is to keep the distribution path for the ozone as smooth as possible so that the ozone moves over an essentially low resistance path with no sharp bends, to minimise the rate of decomposition of the ozone. As already mentioned, the use of a continuous circuit system helps to minimise any back pressure.

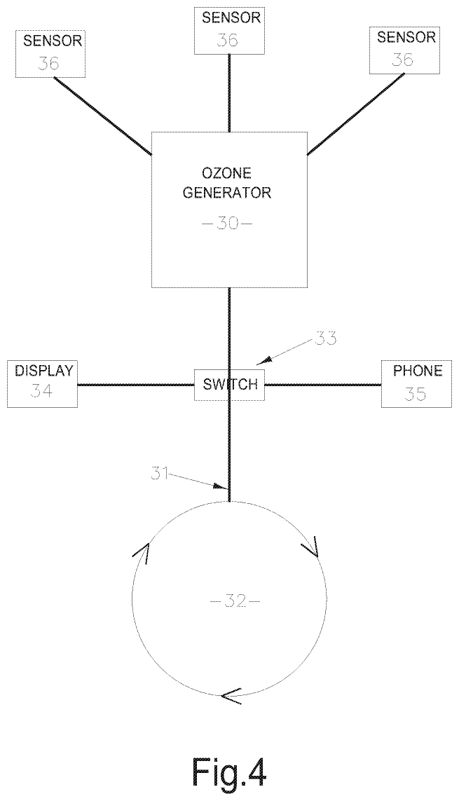

FIG. 4 depicts a typical control system for the system of the present invention. An ozone generator 30 supplies ozone to an outlet pipe 31 which is in turn connected to a distribution circuit 32. A pressure switch 33 is located between the generator 30 and the circuit 32, to check the pressure of ozone being supplied to the circuit. The pressure switch is connected to a manual display 34 for on-the-spot control and also is connected by a wireless connection to the cellphone 35 of the supervising engineer, so that any significant drop in ozone pressure is reported to the supervisor at any time of the day or night.

The system also is provided with a plurality of ozone sensors 36, which may be connected to any suitable read-out and/or may be wired to or wirelessly connected to the generator 30.

The sensors 36 are of known type. Three of the sensors 36 are shown, but in practice a large number of sensors could be used, so that the ozone level could be measured in a number of different parts of the area being treated.

One purpose of the sensors 36 is to establish that the concentration of ozone is sufficiently high in the relevant areas of the building, so that there is an effective amount of ozone in any of the areas of the building where ozone treatment is required.

The other purpose of the sensors 36 is to check that, in the areas where human beings are working, the ozone concentration does not exceed the permitted legal maximum; typically, this is 100 parts per billion for human beings working a continuous eight-hour period in that area.

Each of the sensors 36 is set at the required level for the particular area, and triggers an alarm if either the level of ozone drops below the effective treatment concentration in an area where treatment is required, or if the level of ozone approaches the permitted legal maximum in the areas where human beings are working. The alarm may be shown on a display on the generator 30 and/or may be wirelessly communicated to the supervising engineer's cellphone 35.

The above described system may be used for cool store treatment to reduce bacterial damage, fungal damage, and to retard the ripening of the fruit and vegetables. Ripening fruit and vegetables produce ethylene, and the production of ethylene in itself encourages ripening. Thus, reducing ethylene levels to below 30 ppb in cool stores is advantageous. One means of doing this is to cycle the cool store air through an ethylene scrubber, but ethylene scrubbers are expensive. The system of the present invention enables ethylene to be effectively removed to a low level without needing to use a scrubber, because ozone oxidises ethylene.

For cool store use, the control system of the present invention has an ethylene sensor and a carbon dioxide sensor connected to the control system. If the level of ethylene is reduced, the level of carbon dioxide reduces as well, so the cool store operative needs to know whether either of those two gases is increasing in concentration. A cool store system also typically would include a temperature and a humidity sensor, for a comprehensive control of the system.

In addition to use in cool stores, it has been found that the system of the present invention is unexpectedly effective in repelling birds from the area being treated. Large sheds, animal housing, food storage areas, malls, and supermarkets are very attractive to birds because food is readily available, and such areas may also provide roosts and nesting sites. This can create a very considerable health hazard because of the bird droppings. In addition, in the case of sheds housing animals, birds may eat a significant proportion of the available animal food.

Birds are highly mobile, very persistent and are extremely difficult to exclude from an area. In the case of open sheds, or supermarkets with constantly opening doors, it is virtually impossible to exclude birds. An additional complication is that simply killing the birds is an unpopular option, and in many countries is illegal. The problem therefore remains of finding a method of discouraging birds from entering an area, and also discouraging them from lingering in that area to feed, roost or nest.

It has been found that a low ozone concentration (well below the permitted safe level for human beings) has a rapid and significant repellent effect on birds, due to the design of their respiratory system. It follows that treating the upper part of an area with ozone, using the distribution system of the present invention, is very effective in keeping birds out of the area:—the ozone does not kill the birds, but they find the ozone very unpleasant and it is increasingly difficult for them to breathe, so they move away as rapidly as possible. Naturally birds tend to be in the upper part of any given building, near the ceiling, so providing an ozone distribution system in accordance with the present invention round the upper part of the walls and the ceiling of a building effectively excludes birds from that building without doing any permanent damage to the birds or presenting any hazard to human beings or to larger non-human animals in the area.

The ozone concentration sufficient to repel birds from an area obviously depends on the type of birds which have to be repelled and the conditions in the area (e.g. pressure, temperature, humidity). However, typically an ozone concentration of between 60 and 80 ppb has been found to be sufficient to repel birds effectively from an area.

1. An ozone distribution system into air wherein an ozone generator of known type in use supplies ozone at a preselected concentration to an ozone distribution pipe which is arranged to pass through or around an air-filled area, the air in which is to be treated with ozone, said ozone distribution pipe being arranged to provide a continuous circuit without any closed ends, and to provide a smooth path without sharp bends, said ozone distribution pipe being perforated at intervals along its length, to provide at least one perforated portion through which in use ozone can disperse from the pipe.

2. (canceled)

3. The ozone distribution system as claimed in claim 1, wherein any bend in the continuous circuit is at least 130 degrees.

4. The ozone distribution system as claimed in claim 1, wherein said ozone distribution pipe is arranged to provide two or more vertically separated circuits for ozone distribution, said circuits being connected so as to provide a continuous circuit for circulation of the ozone.

5. The ozone distribution system as claimed in claim 1, wherein said system also includes a pressure switch which located between the ozone generator and the at least one perforated portion of the distribution pipe, to measure the pressure of ozone being supplied to the system.

6. The ozone distribution system as claimed in claim 1, wherein the system also includes one or more ozone sensors distributed around the area to be treated with ozone, the one or more ozone sensors being adapted to indicate the concentration of ozone present in the area immediately adjacent the sensor.

7. The ozone distribution system as claimed in claim 6, wherein the system includes a plurality of ozone sensors; some of the sensors are located around an upper part of the area being treated with ozone, and are arranged in use to trigger an alarm if an ozone level recorded by any of the sensors in said upper part falls below a predetermined level; some of the sensors are located around a lower part of the area being treated with ozone, and are arranged in use to trigger an alarm if an ozone level recorded by any of the sensors in said lower part exceeds a predetermined level.
